(19) United States
(12) Patent Application Publication
(10) Pub. No.: US 2008/0051752 A1
(43) Pub. Date: Feb. 28, 2008

(54) VACUUM-PACKAGED DIAPER APPARATUS AND METHODS

(76) Inventor: Nadia Corlett, Laguna Beach, CA (US)

Correspondence Address: TILLMAN WRIGHT, PLLC, PO BOX 471581, CHARLOTTE, NC, 28247, US

(21) Appl. No.: 11/928,786

(22) Filed: Oct. 30, 2007

Related U.S. Application Data
(63) Continuation-in-part of application No. 11/421,754, filed on Jun. 1, 2006.
Application No. 11/421,754 is a continuation-in-part of application No. 09/560,246, filed on Apr. 26, 2000.

(51) Int. Cl. A61F 13/15

(52) U.S. Cl. 604/385.02; 604/385.201

(57) ABSTRACT

A packaged diaper that is compact and portable comprises a diaper that is vacuum-packaged. The packaged diaper is dimensioned to fit within the pocket of an article of clothing to be worn on one's person. The vacuum-packaged diaper includes packaging made from a substantially flexible, substantially air impermeable material; and a diaper contained within said packaging in a compressed condition. The packaging is hermetically sealed and a negative pressure exists within said packaging such that said diaper is maintained in the compressed condition.

### I. CROSS-REFERENCE TO RELATED APPLICATION

This application is a continuation-in-part of, and claims priority under 35 U.S.C. §120 to, U.S. patent application Ser. No. 11/421,754, filed Jun. 1, 2006, which '754 application published as U.S. patent application publication no. US 2006/0206078 A1, and which '754 application is a continuation-in-part of, and claims priority to, U.S. patent application Ser. No. 09/560,246, filed Apr. 26, 2000. Each of these patent applications and patent publication is hereby incorporated herein by reference.

Additionally, both of U.S. patent application Ser. No. 10/907,389 and its corresponding publication—U.S. patent application publication no. US ______, are hereby incorporated herein by reference.

### II. COPYRIGHT STATEMENT

All of the material in this patent document is subject to copyright protection under the copyright laws of the United States and of other countries. The copyright owner has no objection to the facsimile reproduction by anyone of the patent document or the patent disclosure, as it appears in the Patent and Trademark Office patent file or records, but otherwise reserves all copyright rights whatsoever.

### III. BACKGROUND OF THE PRESENT INVENTION

The present invention relates to absorbent articles such as diapers and sanitary napkins. More particularly, the present invention relates to absorbent articles that are packaged at reduced volume to become ultra-compact and highly portable.

One of the drawbacks of disposable diapers is portability, particularly during travel, whether long-distance travel or simply to the neighborhood store. Parents need to pack a sufficient number of diapers in a diaper bag to last the duration of the trip. However, while not necessarily heavy, disposable diapers are bulky and occupy a large amount of space. Accordingly, a diaper bag in tow with a traveling parent is cumbersome and a nuisance. Indeed, a single disposable diaper may not fit into most purses or a jacket breast pocket even for short trips. In view of the foregoing, there remains a need in the art for absorbent articles that are compact and portable.

### IV. SUMMARY OF THE PRESENT INVENTION

According to one aspect of the invention, a set of compact absorbent articles includes a plurality of absorbent articles and a packaging. Each of the absorbent articles, such as a diaper or a sanitary napkin, is reconfigurable from a normal condition to a compressed condition. The packaging includes a plurality of compartments each for receiving one of the absorbent articles in the compressed condition. Each of the absorbent articles has three dimensions, at least one of which is reduced when the absorbent article is in the compressed condition, with each of the absorbent articles being retained by the packaging when in the compressed condition.

According to various aspects of the invention, the packaging may have release seams disposed between the compartments, so that a user can remove an individual article in its compartment from the other articles in the packaging. Also, each of the absorbent articles may be are vacuumed sealed at negative pressure in the packaging. In addition, the dimension that is reduced may be reduced by at least 30% when the absorbent articles are in the compressed condition. Further, each of the absorbent articles may be reconfigured from the normal condition to the compressed condition by negative pressure, physical force, rolling, or folding.

Regarding advantages, the individually packaged compressed absorbent articles are highly compact and, accordingly, readily portable. In addition, a packaged absorbent article can be easily carried in a purse or a breast pocket of a blazer. In a multipack packaging embodiment, the multipack may be conveniently carried in, e.g., a bag, with individual absorbent articles removed from the multipack as needed.

Additional aspects, features, and advantages of the present invention will become apparent to those skilled in the art from a consideration of the following detailed description taken in conjunction with the accompanying drawings.

### V. BRIEF DESCRIPTION OF THE DRAWINGS

FIG. 1 is a perspective schematic view of an absorbent article of the invention shown in a normal (i.e., uncompressed) condition;

FIG. 2 is a perspective schematic view of the absorbent article of FIG. 1 now shown in a compressed condition;

FIG. 3 is a cross-sectional view of the absorbent article taken along line 3-3 of FIG. 1;

FIG. 4 is a cross-sectional view of the absorbent article taken along line 4-4 of FIG. 2;

FIG. 5 is a perspective view of a reconfigured absorbent article, wherein the article has been rolled in accordance with a method of the invention and is illustrated in a rolled configuration;

FIG. 6 is a perspective view of a reconfigured absorbent article, wherein the article has been folded in accordance with a method of the invention and is illustrated in a folded configuration;

FIG. 7 is a perspective view of the rolled absorbent article of FIG. 5, wherein the article in the rolled configuration has been compressed in accordance with a method of the invention and is illustrated in a compressed condition;

FIG. 8 is a perspective view of the folded absorbent article of FIG. 6, wherein the article in the folded configuration has been compressed in accordance with a method of the invention and is illustrated in a compressed condition;

FIG. 9 is a cross-sectional view of the rolled and compressed absorbent article of FIG. 7, wherein the article is retained by packaging in the rolled and compressed condition;

FIG. 10 is a cross-section view of the folded and compressed absorbent article of FIG. 8, wherein the article is retained by packaging in the folded and compressed condition;

FIG. 11 is a top plan view of a disposable diaper in a “bifold” configuration, wherein the diaper has been folded once about a crosswise fold line along a midsection thereof,

FIG. 12 is a side view of the diaper of FIG. 11;

FIG. 13 is a perspective view of the diaper of FIG. 11;

FIG. 14 is a perspective view of the diaper of FIG. 13 being folded along a lengthwise aspect thereof,

FIG. 15 is a perspective view of the diaper of FIG. 13 being folded along a crosswise aspect thereof,

FIG. 16 is a perspective view of the diaper of FIG. 13 being inserted into a flexible, air impermeable encasement for vacuum 0-packing;

FIG. 17 is a perspective view of the diaper of FIG. 16 after vacuum-packaging;

FIG. 18 is a top plan view of a diaper after folding, compressing, and vacuum-packaging, wherein the diaper is in a “trifold” configuration;

FIG. 19 is a side view of the packaged diaper of FIG. 18;

FIG. 20 is a top plan view of a disposable diaper having indicia thereon, wherein the diaper is completely unfolded;

FIG. 21 and FIG. 22 are views of the diaper of FIG. 20 wherein margins of the diaper have been tucked;

FIG. 23 is a perspective view of the diaper of FIGS. 21 and 22, wherein the diaper has been folded once about a crosswise fold line along a midsection thereof,

FIG. 24 and FIG. 25 are views of the diaper of FIG. 23 after vacuum-packaging;

FIG. 26 is a perspective view of a compressed absorbent article individually packaged in resealable packaging;

FIG. 27 is a schematic plan view of a plurality of individually compressed absorbent articles arranged together in a multipack;

FIG. 28 is a cross-sectional view of the multipack of FIG. 27 taken along the line 32-32;

FIG. 29 is a cross-sectional view of a multipack similar to the multipack of FIG. 27-28, wherein the multipack has been folded back upon itself making the multipack compact and portable;

FIG. 30 is a perspective view of a carrier that is configured to hold and retain a plurality of individually packaged compressed absorbent articles; and

FIG. 31 is a perspective view of a master container that is configured to hold and dispense a plurality of individually packaged compressed absorbent articles.

### VI. DETAILED DESCRIPTION OF PREFERRED EMBODIMENTS

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein-as understood by the Ordinary Artisan based on the contextual use of such term-differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

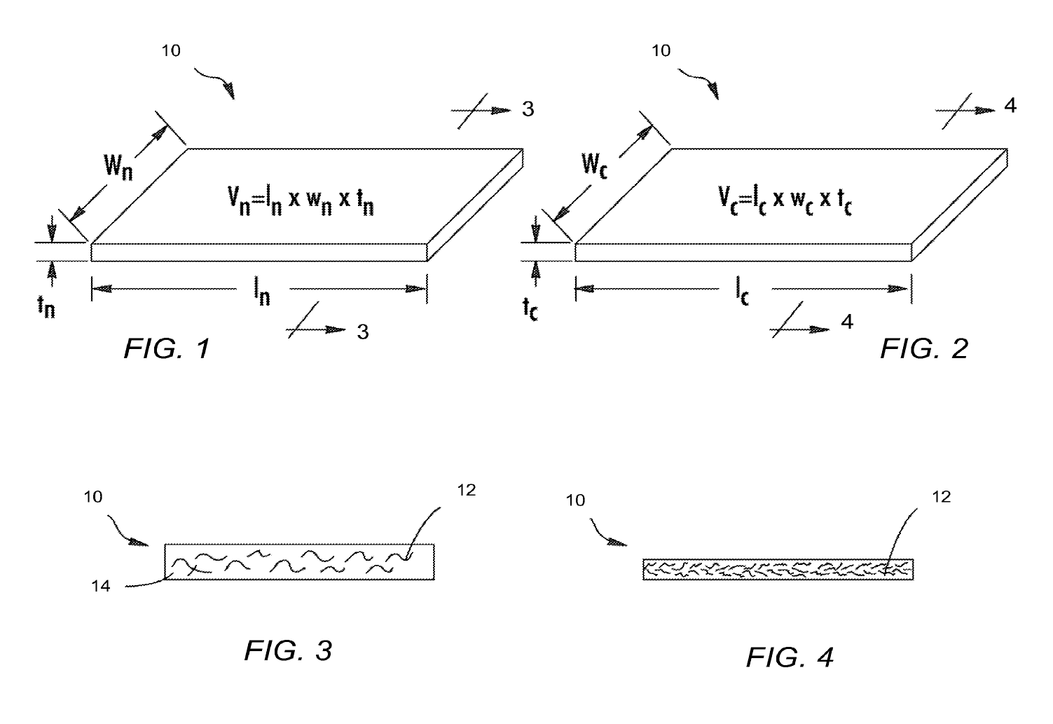

Referring now to the drawings, an absorbent article is shown in FIG. 1 and generally indicated by reference numeral 10. The absorbent article 10, which may be a diaper or a sanitary napkin, is a three-dimensional object occupying a volume of space Vₙ schematically represented by the product of a length lₙ, a width wₙ, and a thickness tₙ, i.e., Vₙ=lₙ times wₙ times tₙ. The absorbent article 10 is compressible in at least one dimension but preferably in all three dimensions so that the volume of space which the article 10 occupies is reduced or compressed.

For example, the absorbent article 10 is shown in FIG. 2 with compressed length, width, and thickness dimensions l_{c}, w_{c}, and t_{c}, each of which is respectively less than the uncompressed or “normal” length, width, and thickness dimensions lₙ, wₙ, and tₙ of FIG. 1. Accordingly, a compressed volume V_{c} of the absorbent article 10 is less than the normal volume Vₙ thereof. For example, in accordance with the present invention, the absorbent article 10 is compressible such that the compressed volume V_{c} is at least one third less and preferably at least one half less than the normal volume Vₙ; that is, the compressed volume V_{c} of the absorbent article is at least 30% less and is preferably at least 50% less than the uncompressed volume Vₙ. In accordance with the principles of the present invention, the absorbent article 10 is retained in the compressed condition, for example, by vacuum packaging, thereby providing an ultra-compact and highly portable absorbent article. The absorbent article 10 retained in the compressed condition may then be easily and conveniently carried by a user until needed.

The absorbent article 10 is illustrated schematically in the drawings but may be configured as any type of absorbent article as known in the art, for example, a diaper, either infant or adult, a feminine sanitary napkin, and so on. The absorbent article 10 is made from resilient and compressible material that is able to retain fluid. Examples of diapers are disclosed in U.S. Statutory Invention Registration No. H1674 and U.S. Pat. Nos. 5,520,674; 5,522,810; 5,855,574; 5,876,393; and 5,980,500. Examples of feminine sanitary napkins are disclosed in U.S. Pat. Nos. 5,490,847; 5,653,702; 5,792,131; 5,797,894; and 6,015,934. The disclosure of each of these patents is incorporated herein by reference.

Being made from resilient absorptive material, exemplary article 10 is compressible in at least one dimension. However, to minimize the amount of compressed volume V_{c}, it is preferable for the absorbent article 10 to be compressible in three dimensions, as mentioned above. More specifically, as shown in FIG. 3, exemplary article 10 includes absorptive material 12 with inter-fiber spaces 14. To reduce at least one of the three dimensions, the absorbent article 10 is compressed, thereby substantially eliminating the inter-fiber spaces 14 and rendering the absorptive material 12 more dense, which condition is shown in FIG. 4.

To compress, the absorbent article 10 may be subject to compressive force, thereby forcing air out of the inter-fiber spaces 14 and compressing the absorptive material 12. Alternatively, the absorbent article 10 may be subject to vacuum, thereby drawing air out of the inter-fiber spaces 14 and compressing the absorptive material 12. Once compressed, the absorbent article 10 is retained to maintain the compact and highly portable configuration, for example, by shrink-wrap thermoplastic packaging at a vacuum or negative pressure, which will be discussed in more detail below. To use, the retaining packaging is disengaged or opened, allowing the absorbent article 10 to return to expand under the resiliency and elasticity of the absorptive material 12, which is also discussed in more detail below.

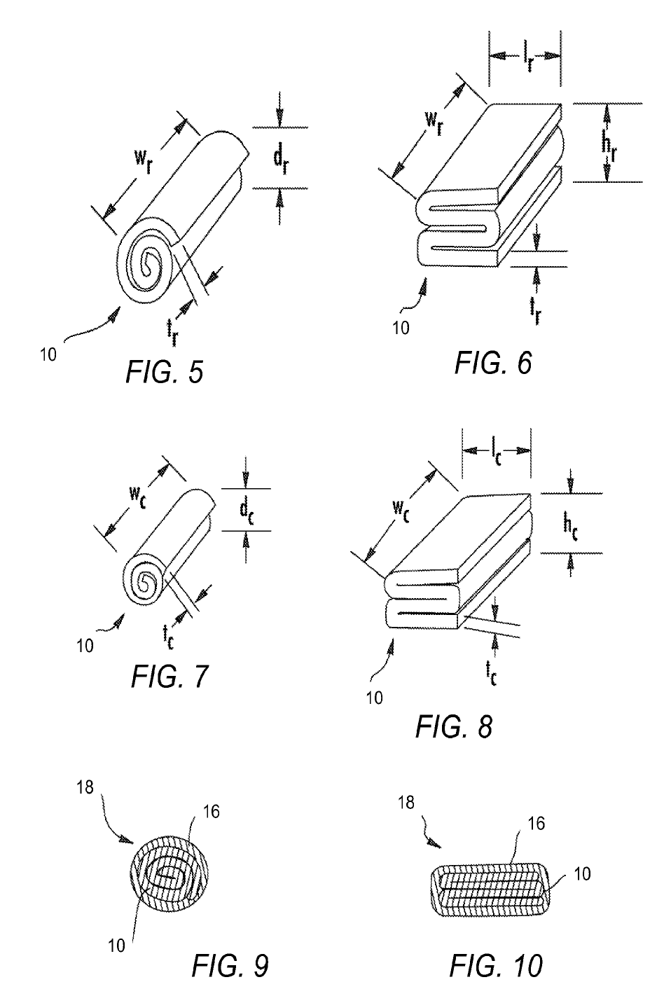

As mentioned above, the absorbent article 10 is shown in a normal condition in FIGS. 1 and 3 in which the article is unstressed, uncompressed, and ready for use. Prior to being placed in the compressed condition as shown in FIGS. 2 and 4, the absorbent article 10 may be reconfigured in any of a variety of ways, many of which will be readily apparent to the Ordinary Artisan. The absorbent article 10 may be reconfigured, for example, by rolling as schematically shown in FIG. 5 or by folding as schematically shown in FIG. 6, the latter sometimes being referred to herein as the “M-folded configuration.” When reconfigured, the absorbent article 10 has at least three dimensions, for example, a width wᵣ, a thickness tᵣ, and a diameter dᵣ as shown in FIG. 5, or a length lᵣ, a width wᵣ, a thickness tᵣ, and a height hᵣ, as shown in FIG. 6.

After being placed in a reconfigured condition, the absorbent article 10 may then be compressed as discussed above, thereby placing the article in a compressed condition. If reconfigured by rolling as shown in FIG. 5, then when compressed the absorbent article 10 takes on the compressed condition shown in FIG. 7 with compressed dimensions of a width w_{c}, a thickness t_{c}, and a diameter d_{c}. If reconfigured by folding as shown in FIG. 6, then when compressed the absorbent article 10 takes on the compressed condition shown in FIG. 8 with compressed dimensions of a length l_{c}, a width w_{c}, a thickness t_{c}, and a height h_{c}. It will likewise be apparent that when reconfigured into a wide variety of other configurations, including several shown and described herein, and then when compressed, each such reconfigured absorbent article 10 will take on a compressed condition with corresponding compressed dimensions. In each case, and analogous to the description above in relation to FIGS. 1 and 2, at least one of the compressed dimensions, but preferably all of the compressed dimensions, of the absorbent article 10 are respectively less than the reconfigured dimensions thereof.

After being compressed, the absorbent article 10 may then be retained to maintain the compressed condition, for example, with packaging 16 as shown in FIGS. 9 and 10, thereby yielding an individually packaged article 18. As mentioned above, the packaging 16 may be a thermoplastic material that allows the compressed absorbent article 10 to be hermetically sealed therein. Alternatively, the packaging 16 may be any other material suitable for retaining the absorbent article 10 in the compressed condition. The packaging 16 ensures that the absorbent article 10 is clean and sanitary when the packaged article 18 is opened for use.

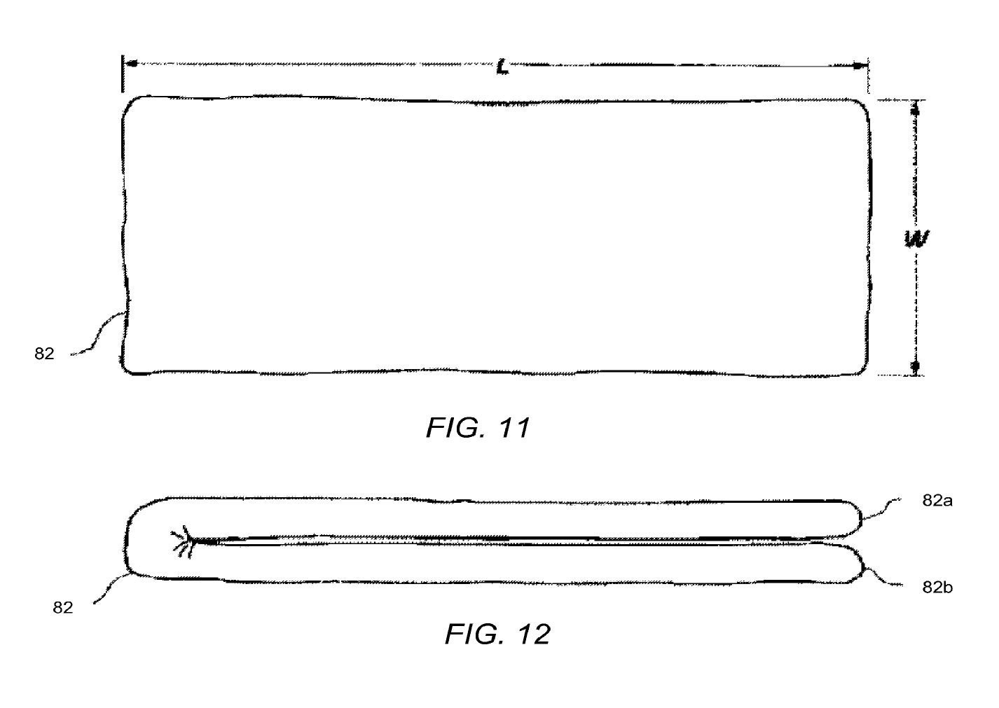

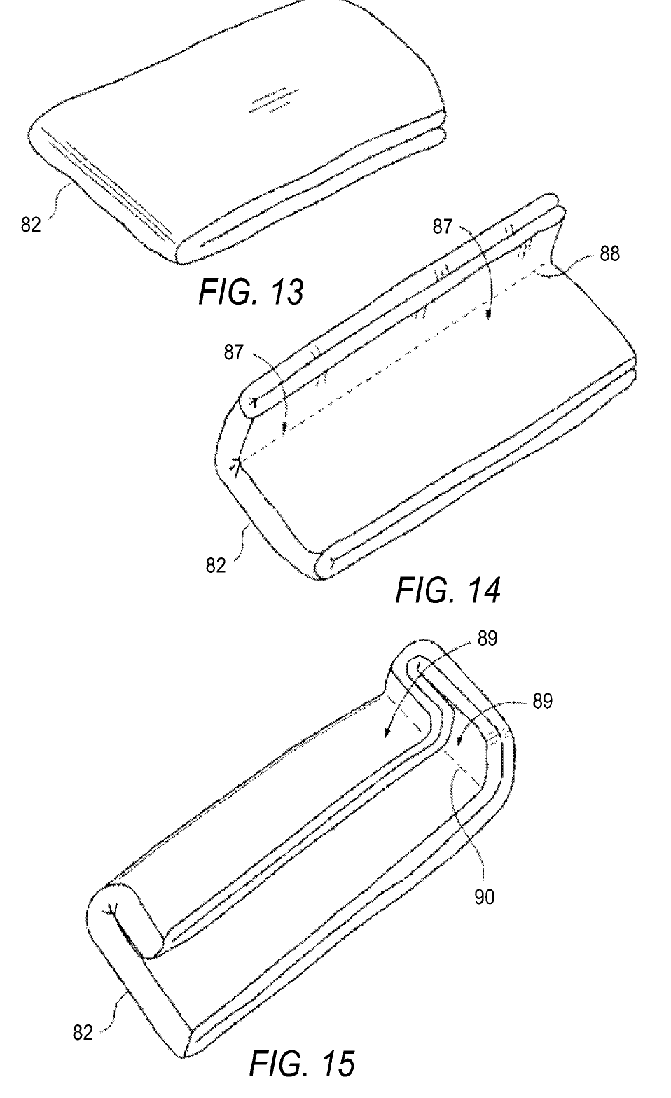

Other examples of how an absorbent article may be reconfigured are illustrated by FIGS. 11-15. In this regard, FIG. 11 is a top view of a diaper 82 that has been folded along a centered, crosswise aspect to obtain a folded rectangular configuration. FIG. 12 is a side view of the diaper 82, and FIG. 13 is a perspective view of the diaper 82. It will be appreciated that, in folding the diaper 82 to arrive at the configuration shown in FIGS. 11-13, a fold line necessarily extends through the absorbent core of the diaper 82. The fold line effectively creates an upper portion 82a and a lower portion 82b, which may correspond to a front and a back of the diaper 82. This configuration is sometimes referred to as the “bifold” configuration. The diaper 82 may be further reconfigured by folding the diaper in the bifold configuration along one or more lengthwise aspect, as shown in FIG. 14, along one or more crosswise aspect, as shown in FIG. 15, or along both. Specifically, folding the diaper 82 along fold line 88, as represented by arrows 87, results in a diaper that is reconfigured along a lengthwise aspect thereof, and folding 89 the diaper 82 along fold line 90, as represented by arrows 89, results in a diaper that is reconfigured along a crosswise aspect thereof. Furthermore, any desired number of lengthwise and/or crosswise folds may be applied to a diaper, as desired, and a diaper may be so folded from any other configuration and is not limited to the bifold configuration shown.

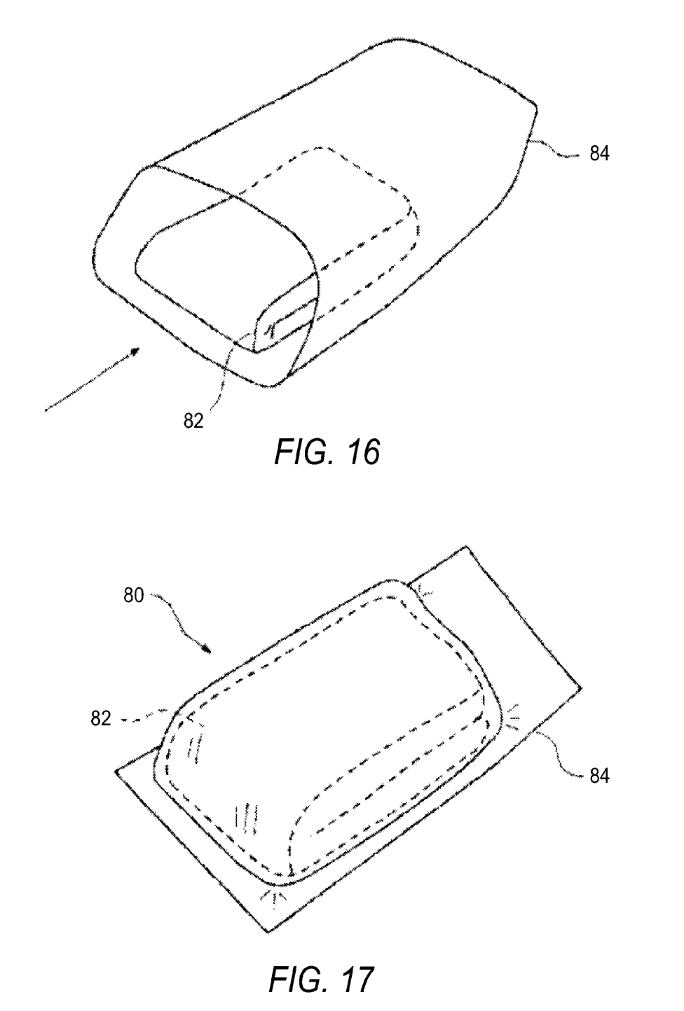

After the diaper 82 is reconfigured, it may be compressed and maintained in the compressed configuration by vacuum-packaging. For example, FIG. 16 illustrates the insertion of the reconfigured diaper 82 within an encasement 84 and FIG. 17 shows the resulting packaged diaper 80 after vacuum-packaging.

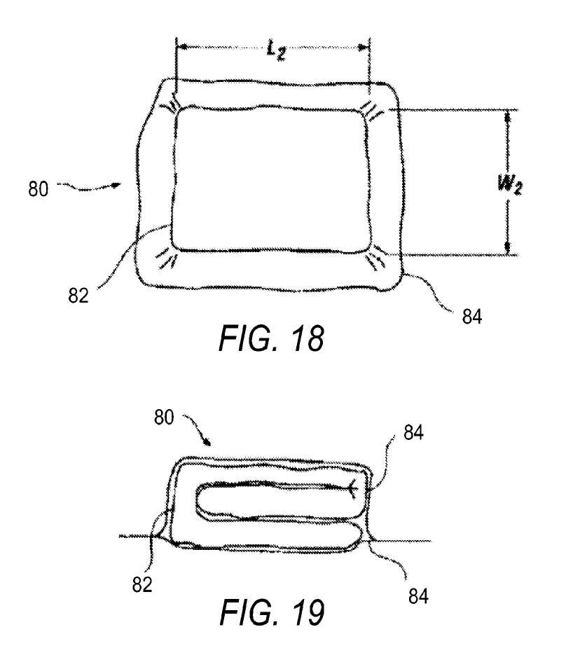

Another example of a diaper 82 that has been reconfigured and then vacuum-packaged within an encasement 84 is shown in FIGS. 18-19. A top view of the vacuum-packaged diaper 80 is shown in FIG. 18, and a side view of the vacuum-packaged diaper 80 is shown in FIG. 19. The vacuum-packaged diaper 80 includes a length L.sub.2 that is less than the length L of the diaper in the bifold configuration shown in FIG. 11, and a width W.sub.2 that is less than the width W of the diaper in the bifold configuration shown in FIG. 11. The diaper of FIGS. 18-19 has been folded twice and thus includes two fold lines that extend along a crosswise aspect of the diaper. This configuration of the diaper is sometimes referred to as a “trifold” configuration.

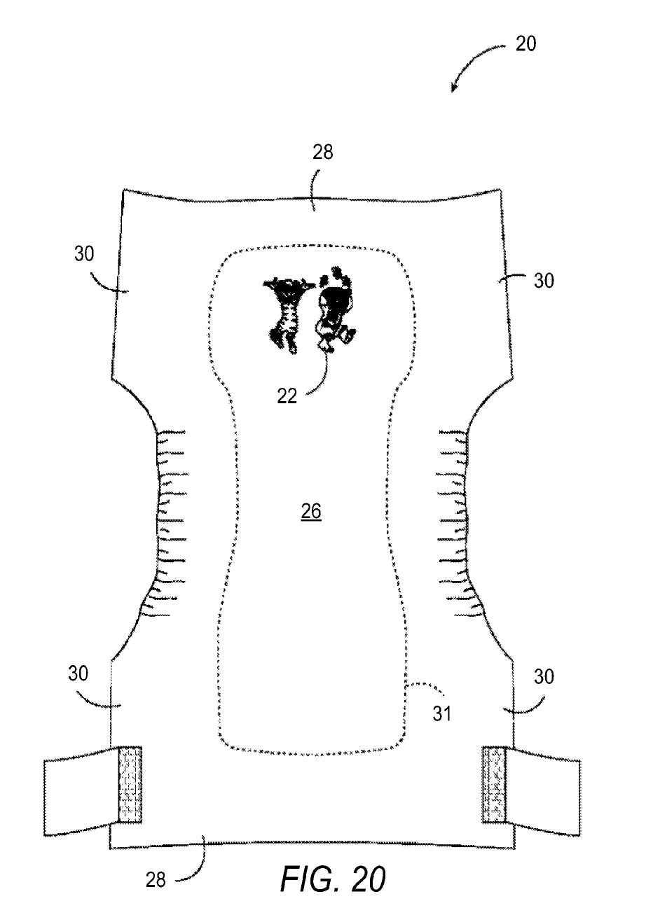

Yet another example of reconfiguring a diaper and then packaging the diaper is illustrated by FIGS. 20-25. In this respect, FIG. 20 illustrates a diaper 20 without any folds. The fully extended diaper 20 shown in FIG. 20 includes indicia 22 printed on a front portion of an outer surface 26 of the diaper 20. When printed on this portion, the indicia 22 is situated proximal the groin area and navel of the wearer when the diaper 20 of FIG. 20 is worn.

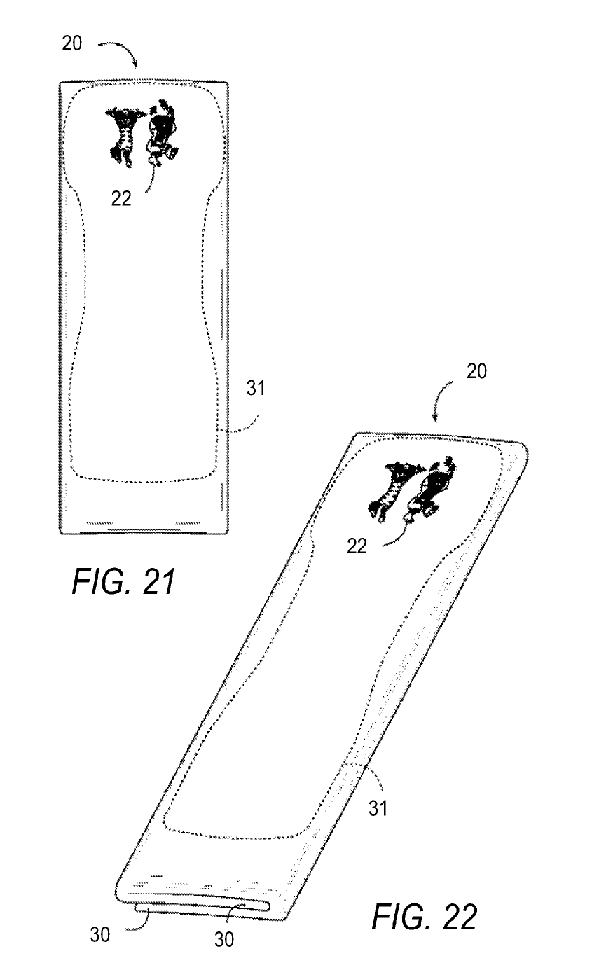

FIGS. 21 and 22 illustrate the diaper 20 of FIG. 20 after margins of the diaper have been tucked. The “margins” of a diaper generally includes the end margins 28 and side margins 30 and include such items as elastics, tabs, and the like. Tucking of the margins results in the unfolded, rectangular configuration of the diaper shown in FIGS. 21 and 22. It will be noted that no fold line extends through the area of absorbent material of the diaper 20, which area also may be referred to as the “absorbent core,” and which area of the diaper is indicated by dashed line 31.

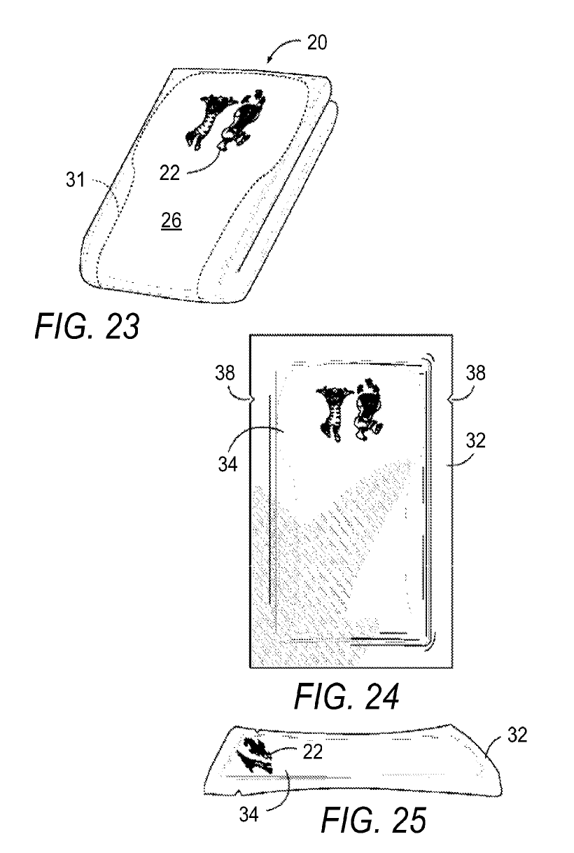

From the rectangular configuration of the diaper 20 shown in FIGS. 21 and 22, the diaper 20 is folded along a centered, crosswise aspect to obtain a folded rectangular configuration, such as shown in FIG. 23. Furthermore, it will be appreciated that, in folding the diaper 20 to arrive at the configuration shown in FIG. 23, a fold line necessarily extends through the absorbent core 31 of the diaper 20.

The diaper 20 may then be vacuum-packaged within an air and moisture impermeable encasement 32. The resulting vacuum-packaged diaper is illustrated in FIGS. 24 and 25. At least a portion of the encasement 32 is transparent such that the indicia 22 printed on the surface 26 of the diaper is viewable through the encasement 32. Additionally, notches 38 are formed along outer edges of the encasement 32 to aid in opening the encasement 32.

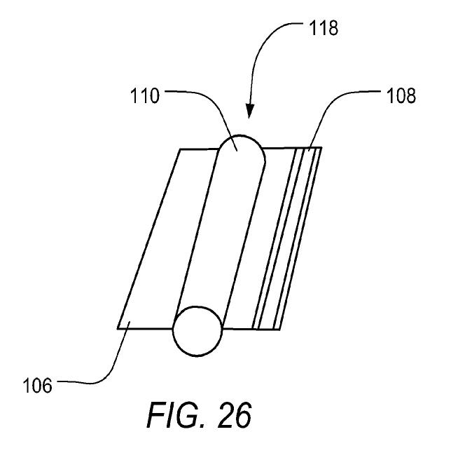

As shown in FIG. 26, individually packaged compressed absorbent articles 118 may include sealable packaging 106. More specifically, exemplary packaging 106 may include a resealable closure mechanism 108. Accordingly, a user may open the packaging 106 and remove the absorbent article 110, thereby allowing the compressed absorbent article to regain the normal condition for use. The now-empty packaging 106 then may receive and store a soiled absorbent article to be discarded in the future.

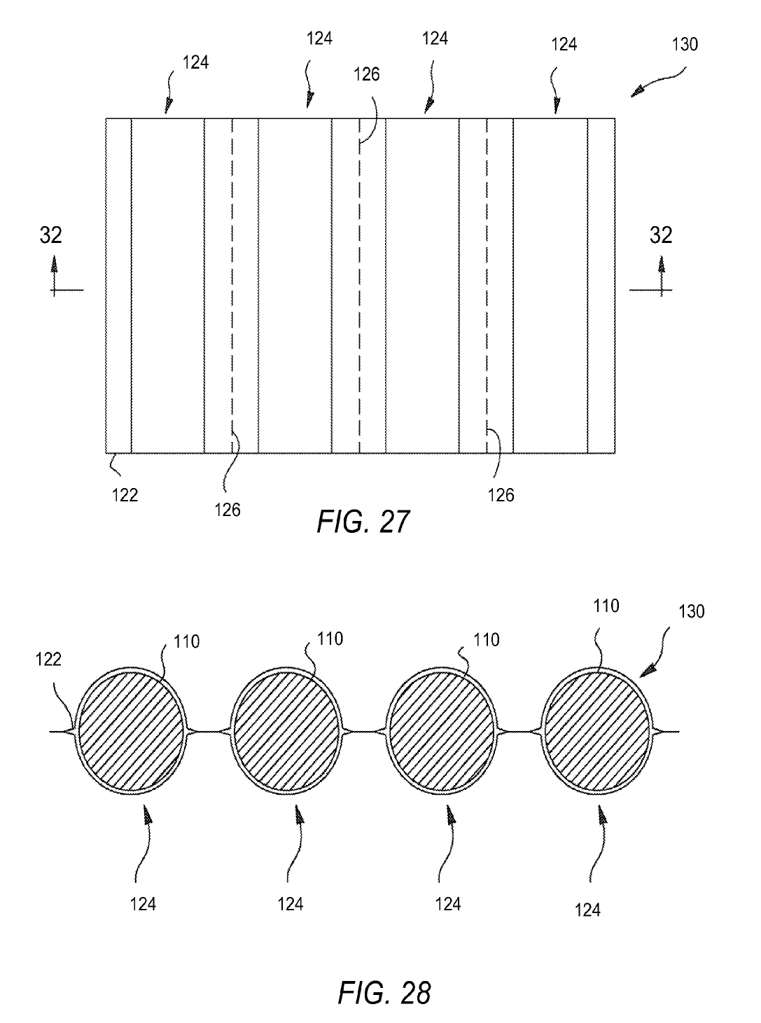

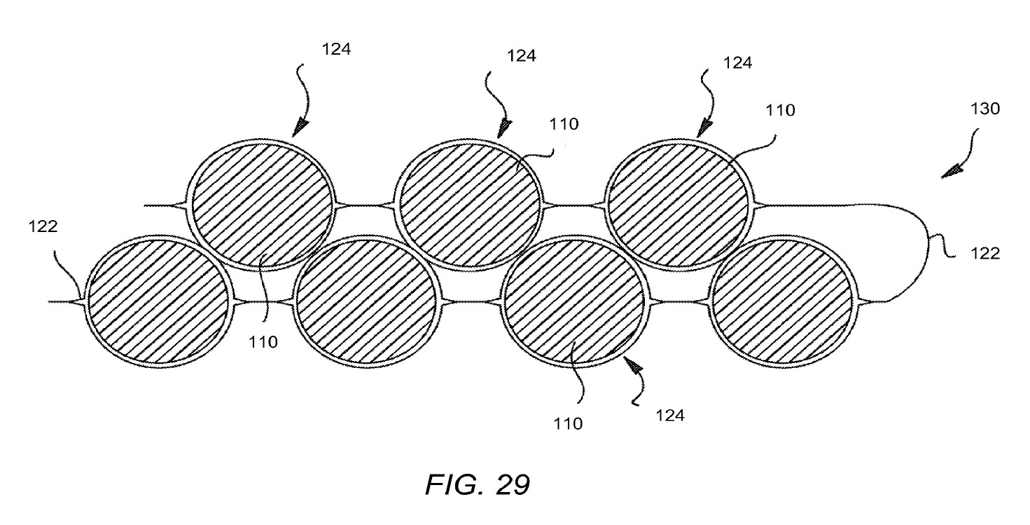

In addition to individually compressed and packaged absorbent articles such as articles 18, 80, 118, a plurality of compressed absorbent articles may be packaged together in various multipack arrangements. One multipack of individually compressed and packaged absorbent articles 110 is shown in FIGS. 27 and 28, wherein the multipack is indicated by reference numeral 130. The multipack 130 of articles includes packaging 122 for retaining a plurality of absorbent articles 110 in the compressed condition. The absorbent articles 110 are configured within the packaging 122 such that each article 110 is individually sealed, which is particularly shown in FIG. 28 and indicated by reference numeral 124. The packaging 122 includes perforations 126 between the individually sealed articles 124 so that a user may remove articles 124 as needed. In addition, the packaging 122 may be substantially flexible so that the multipack 130 may be folded back onto itself to be more compact and portable as shown in FIG. 29.

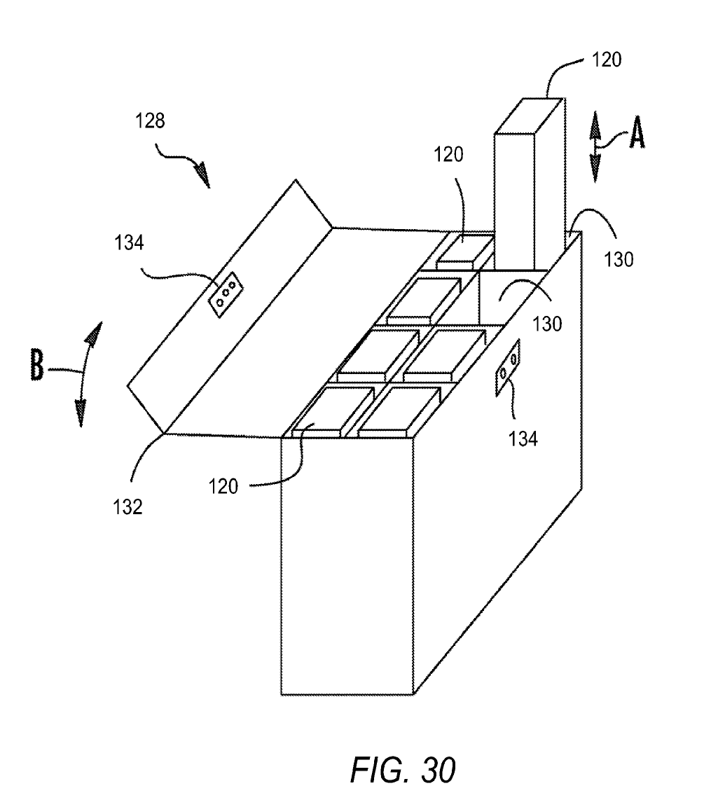

Another example of a multipack of individually compressed and packaged absorbent articles is shown in FIG. 30, wherein each of a plurality of containers 120 are arranged in a carrier 128 for convenient transport. Exemplary carrier 128 includes a plurality of compartments 130 each configured to receive a container 120 as shown by arrow A. The carrier 128 may also include a hinged cover 132 which pivots as shown by arrow B. The carrier 128 may also include releasable fastening means 134, such as complementary hook-and-eye fasteners 134, for retaining the cover 132 in a closed condition on the carrier. Each container 120 may be rectangular in shape, as shown, and includes therein an individually compressed and packaged absorbent article in accordance with the invention.

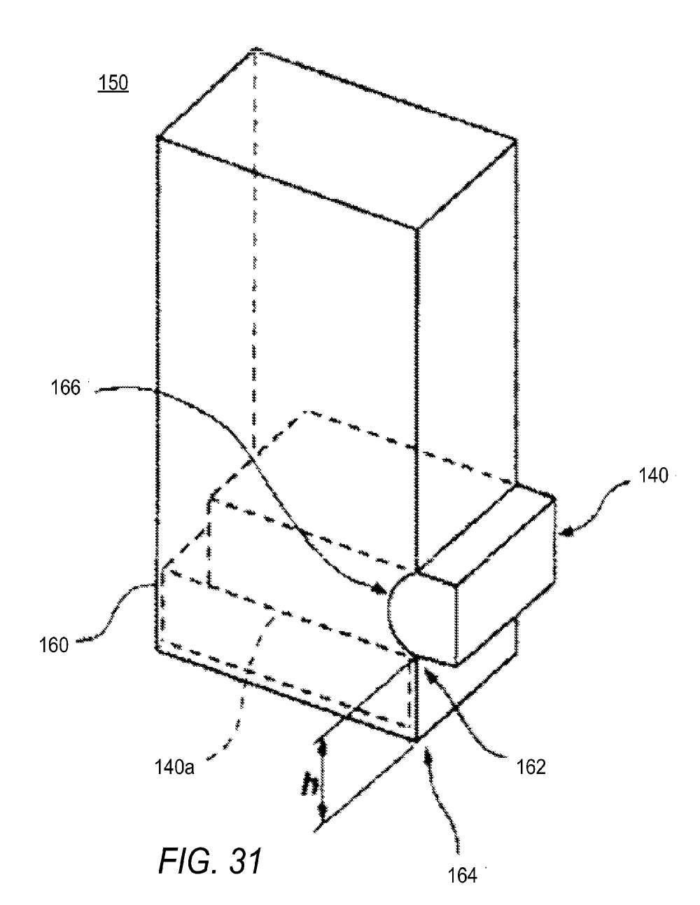

Another example of a multipack 150 of individually compressed and packaged absorbent articles is shown in FIG. 31, wherein each of a plurality of containers are arranged in a vertically stacked configuration within a master container 160. Each container may be rectangular in shape, as shown, and includes therein an individually compressed and packaged absorbent article. The master container 160 includes a removable access panel (previously removed and not shown) through which one 140 of the plurality of containers is accessed. The removable access panel may be perforated to facilitate its removal from the master container 160. A bottom 162 of the access panel can be disposed in the master container 160 in a location vertically displaced from a bottom 164 of the master container 160 by a distance substantially equal to a height “h” of a container 140. In this manner, as a container 140 is removed from the master container 160, the container (not shown) immediately above the removed container 140 will fall into place and will then be easily removable from the master container 160. Notches 166 are provided adjacent the removable panel to enable easy withdrawal of container 140 through the opening. The master container 160 can be used to package and ship a number of containers 140 and, advantageously, can be used to dispense containers 140 at, for example, a retail center or other location. The master container 160 shown in FIG. 31 may be configured to store and dispense any desired number of containers 140, and may be configured to include two or more access panels disposed and oriented in a variety of locations on the master container 160, as desired.

What is claimed is:

1. An individually packaged diaper that is compact and portable, comprising packaging made from a substantially air impermeable material and a diaper that is individually vacuum-packaged in said packaging such that said diaper is maintained in a compressed condition.

2. A packaged diaper that is compact and portable, comprising packaging made from a substantially air impermeable material and a vacuum-packaged diaper that is maintained in a compressed condition within said packaging and dimensioned to fit within the pocket of an article of clothing to be worn on one's person.

3. An individually packaged diaper that is compact and portable, comprising:
(a) packaging made from a substantially flexible, substantially air impermeable material; and
(b) a diaper contained within said packaging in a compressed condition;
(c) wherein said packaging is hermetically sealed and a negative pressure exists within said packaging such that said diaper is maintained in said compressed condition.

4. The packaged diaper of claim 3, wherein said diaper is the only diaper contained within said packaging.

5. The packaged diaper of claim 3, wherein said diaper is dimensioned to fit within the pocket of an article of clothing on one's person.

6. The packaged diaper of claim 5, wherein said diaper is dimensioned to fit within a pocket of a jacket that is worn on one's person.

7. The packaged diaper of claim 3, wherein said material comprises a thermoplastic shrink-wrap material.

8. The packaged diaper of claim 3, wherein said diaper is disposed within said packaging in a folded arrangement and has three crosswise folds.

9. The packaged diaper of claim 8, wherein said three crosswise folds are about evenly spaced along a length of the diaper between opposed ends of the diaper.

10. The packaged diaper of claim 3, wherein said diaper is disposed within said packaging in a rolled arrangement.

11. The packaged diaper of claim 3, wherein a volume of said diaper in said compressed condition is reduced relative to a volume of said diaper when uncompressed.

12. The packaged diaper of claim 11, wherein the reduction is by at least 30%.

13. The packaged diaper of claim 11, wherein the reduction is by at least 50%.

14. The packaged diaper of claim 3, wherein two dimensions of the group of height, width, and length of said diaper in said compressed condition are reduced relative to the corresponding height, width, and length of said diaper when uncompressed.

15. The packaged diaper of claim 14, wherein the reduction of a dimension is by at least 30%.

16. A method for vacuum-packaging a single disposable diaper to yield a single individually packaged disposable diaper that is compact and portable, comprising the steps of:
(a) compressing the disposable diaper from a first condition to a second, compressed condition such that each of at least two dimensions of the disposable diaper is reduced, and such that at least one of the reduced dimensions is reduced by at least 30%, said step of compressing being performed without folding of the disposable diaper and without rolling of the disposable diaper; and
(b) retaining the disposable diaper in the second, compressed condition by,
(i) locating the diaper in an interior space defined by a substantially flexible material,
(ii) creating a negative pressure within the interior space, and
(iii) hermetically sealing the diaper at the negative pressure within the interior space of the flexible material such that a pressure differential acts upon the flexible material to maintain the diaper in the compressed condition;
(c) wherein the disposable diaper is the only diaper located within the interior space of the flexible material.

17. The method of claim 16 wherein the flexible material comprises a thermoplastic shrink-wrap material.

18. The method of claim 16 further comprising, prior to said step (a) of compressing the disposable diaper, reconfiguring the diaper.

19. The method of claim 18 wherein said step of reconfiguring the disposable diaper includes folding the diaper.

20. The method of claim 19 wherein said step of folding includes forming three widthwise folds in the disposable diaper.

21. The method of claim 21, wherein said three widthwise folds are about evenly spaced along a length of the diaper between opposed ends of the diaper.

22. The method of claim 18, wherein said step of reconfiguring the disposable diaper includes rolling the diaper such that the disposable diaper is cylindrical in shape.

23. The method of claim 16, wherein said step (a) of compressing the disposable diaper includes reducing each of height, width, and length dimensions of the disposable diaper.

24. The method of claim 16, wherein said step (a) of compressing the disposable diaper includes reducing each of width, thickness, and diameter dimensions of the disposable diaper.

25. The method of claim 16, wherein said step (a) of compressing the disposable diaper includes compressing the diaper such that a volume of the diaper is reduced by at least 30%.

26. The method of claim 16, wherein said step (a) of compressing the disposable diaper includes compressing the diaper such that a volume of the diaper is reduced by at least 50%.

27. A method for vacuum-packaging a single disposable diaper to yield a single individually packaged disposable diaper that is compact and portable, comprising the steps of:
(a) compressing the disposable diaper from a first condition to a second, compressed condition such that each of at least two dimensions of the disposable diaper is reduced, and such that a volume of the disposable diaper is reduced by at least 30%, said step of compressing being performed without folding of the disposable diaper and without rolling of the disposable diaper; and
(b) retaining the disposable diaper in the second, compressed condition by,
(i) locating the diaper in an interior space defined by a substantially flexible material,
(ii) creating a negative pressure within the interior space, and
(iii) hermetically sealing the diaper at the negative pressure within the interior space of the flexible material such that a pressure differential acts upon the flexible material to maintain the diaper in the compressed condition;
(c) wherein the disposable diaper is the only diaper located within the interior space of the flexible material.

28. The method of claim 27, wherein said step (a) of compressing the disposable diaper includes compressing the diaper such that a volume of the diaper is reduced by at least 50%.

29. The method of claim 27, wherein said step (a) of compressing the disposable diaper includes compressing the diaper such that at least one of the reduced dimensions is reduced by at least 30%.

30. The method of claim 27, wherein the flexible material comprises a thermoplastic shrink-wrap material.

31. A method for vacuum-packaging a single disposable diaper to yield a single individually packaged disposable diaper that is compact and portable, comprising the steps of:
(a) first, folding the disposable diaper;
(b) after said folding step, compressing the disposable diaper from a first condition to a second, compressed condition such that each of three dimensions of the disposable diaper is reduced, and such that at least one of the reduced dimensions is reduced by at least 30%, said step of compressing being performed without folding of the disposable diaper and without rolling of the disposable diaper; and
(c) retaining the disposable diaper in the second, compressed condition by,
(i) locating the diaper in an interior space defined by a substantially flexible material,
(ii) creating a negative pressure within the interior space, and
(iii) hermetically sealing the diaper at the negative pressure within the interior space of the flexible material such that a pressure differential acts upon the flexible material to maintain the diaper in the compressed condition;
(d) wherein the disposable diaper is the only diaper located within the interior space of the flexible material.

32. The method of claim 31, wherein said step (a) of compressing the disposable diaper includes compressing the diaper such that a volume of the diaper is reduced by at least 50%.

33. The method of claim 31, wherein said step (a) of folding includes forming three widthwise folds in the disposable diaper.

34. The method of claim 33, wherein the three widthwise folds are about evenly spaced along a length of the diaper between opposed ends of the diaper.

35. The method of claim 31, wherein the flexible material comprises a thermoplastic shrink-wrap material.

36. A method of traveling with a disposable diaper on one's person, comprising the steps of:
(a) first, packaging a disposable diaper such that the packaged diaper is dimensioned to fit within a pocket of an article of clothing on one's person, said packaging including,
(i) compressing the disposable diaper from a first condition to a second, compressed condition, and
(ii) retaining the disposable diaper in the second, compressed condition by,
(A) locating the diaper in an interior space defined by a substantially flexible material,
(B) creating a negative pressure within the interior space, and
(C) hermetically sealing the diaper at the negative pressure within the interior space of the flexible material such that, when placed within a pocket of an article of clothing on one's person, a pressure differential acts upon the flexible material to maintain the diaper in the compressed condition; and
(b) thereafter,
(i) placing said packaged diaper in a pocket of an article of clothing on one's person, and
(ii) traveling with said disposed packaged diaper in the pocket of the article of clothing on one's person.

37. The method of claim 36, wherein said packaged diaper is dimensioned to fit within a pocket of a jacket that is worn on one's person.

38. The method of claim 36, wherein said packaged diaper is the only diaper contained within the interior space of the flexible material.

39. The method of claim 36, wherein the flexible material comprises a thermoplastic shrink-wrap material.

40. The method of claim 36, wherein said step (a) of packaging the disposable diaper further includes, prior to said compressing, reconfiguring the diaper.

41. The method of claim 40, wherein said step of reconfiguring the disposable diaper includes folding the diaper.

42. The method of claim 41, wherein said folding includes forming three widthwise folds in the disposable diaper.

43. The method of claim 42, wherein said three widthwise folds are about evenly spaced along a length of the diaper between opposed ends of the diaper.

44. The method of claim 40, wherein said step of reconfiguring the disposable diaper includes rolling the diaper such that the disposable diaper is cylindrical in shape.

45. The method of claim 36, wherein said compressing of the disposable diaper from a first condition to a second includes compressing the diaper in a normal condition without folding the diaper.

46. The method of claim 36, wherein said compressing of the disposable diaper includes reducing at least two of height, width, and length dimensions of the disposable diaper.

47. The method of claim 46, wherein at least two of the height, width, and length dimensions of the disposable diaper are reduced without folding or rolling of the diaper during said compressing.

48. The method of claim 47, wherein at least one of the reduced dimensions is reduced by at least 30%.

49. The method of claim 36, wherein said compressing of the disposable diaper includes reducing each of height, width, and length dimensions of the disposable diaper.

50. The method of claim 49, wherein the height, width, and length dimensions of the disposable diaper are reduced without folding or rolling of the diaper during said compressing.

51. The method of claim 50, wherein at least one of the reduced dimensions is reduced by at least 30%.

52. The method of claim 36, wherein said compressing of the disposable diaper includes reducing at least two of width, thickness, and diameter dimensions of the disposable diaper.

53. The method of claim 52, wherein at least two of the width, thickness, and diameter dimensions of the disposable diaper are reduced without folding or rolling of the diaper during said compressing.

54. The method of claim 53, wherein at least one of the reduced dimensions is reduced by at least 30%.

55. The method of claim 36, wherein said compressing of the disposable diaper includes reducing each of width, thickness, and diameter dimensions of the disposable diaper.

56. The method of claim 55, wherein the width, thickness, and diameter dimensions of the disposable diaper are reduced without folding or rolling of the diaper during said compressing.

57. The method of claim 56, wherein at least one of the reduced dimensions is reduced by at least 30%.

58. The method of claim 36, wherein said compressing of the disposable diaper includes compressing the diaper such that a volume of the diaper is reduced by at least 30%.

59. The method of claim 36, wherein said compressing of the disposable diaper includes compressing the diaper such that a volume of the diaper is reduced by 50%.

60. A method of traveling with a disposable diaper on one's person, comprising the steps of:
(a) first, packaging a disposable diaper such that the packaged diaper is dimensioned to fit within a pocket of an article of clothing on one's person, said packaging including,
(i) folding the disposable diaper by forming three widthwise folds in the disposable diaper evenly spaced along a length of the diaper between opposed ends of the diaper,
(ii) after said folding,
(A) compressing the disposable diaper from a first condition to a second, compressed condition such that each of the height, width, and length dimensions of the disposable diaper is reduced without folding of the diaper during said compressing, and such that at least one of the reduced dimensions is reduced by at least 30%, and
(B) retaining the disposable diaper in the second, compressed condition by,
(1) locating the diaper in an interior space defined by a substantially flexible material,
(2) creating a negative pressure within the interior space, and
(3) hermetically sealing the diaper at the negative pressure within the interior space of the flexible material such that, when placed within a pocket of an article of clothing on one's person, a pressure differential acts upon the flexible material to maintain the diaper in the compressed condition; and
(b) thereafter,
(i) placing said packaged diaper in a pocket of an article of clothing on one's person, and
(ii) traveling with said disposed packaged diaper in the pocket of the article of clothing on one's person.

61. A method of traveling with a disposable diaper on one's person, comprising the steps of:
(a) first, packaging a disposable diaper such that the packaged diaper is dimensioned to fit within a pocket of an article of clothing on one's person, said packaging including,
(i) rolling the disposable diaper such that the disposable diaper is cylindrical in shape,
(ii) after said rolling,
(A) compressing the disposable diaper from a first condition to a second, compressed condition such that each of the width, thickness, and diameter dimensions of the disposable diaper is reduced without rolling of the diaper during said compressing, and such that at least one of the reduced dimensions is reduced by at least 30%, and
(B) retaining the disposable diaper in the second, compressed condition by,
(1) locating the diaper in an interior space defined by a substantially flexible material,
(2) creating a negative pressure within the interior space, and
(3) hermetically sealing the diaper at the negative pressure within the interior space of the flexible material such that, when placed within a pocket of an article of clothing on one's person, a pressure differential acts upon the flexible material to maintain the diaper in the compressed condition; and
(b) thereafter,
(i) placing said packaged diaper in a pocket of an article of clothing on one's person, and
(ii) traveling with said disposed packaged diaper in the pocket of the article of clothing on one's person.

62. A method of traveling with a disposable diaper on one's person, comprising the steps of:
(a) first, vacuum-packaging a disposable diaper such that the vacuum-packaged diaper is dimensioned to fit within a pocket of an article of clothing on one's person, said vacuum-packaged diaper comprising,
(i) packaging made from a substantially flexible material, and
(ii) a disposable diaper contained within the packaging in a compressed condition,
(iii) wherein the packaging is hermetically sealed at negative pressure such that a pressure differential acts upon the packaging to maintain the diaper in the compressed condition; and
(b) thereafter,
(i) placing said vacuum-packaged diaper in a pocket of an article of clothing on one's person, and
(ii) traveling with said disposed packaged diaper in the pocket of the article of clothing on one's person.

63. A method of packaging a diaper, comprising vacuum-packaging a diaper such that the diaper is compressed and retained in the compressed condition, and such that the packaged diaper is dimensioned to fit within the pocket of an article of clothing to be worn on one's person.

64. The method of claim 63, wherein the diaper is vacuum-packaged within an interior space of a substantially flexible, substantially air impermeable packaging material and is the only diaper contained within an interior space of the packaging material.

65. A method of individually packaging a diaper, comprising vacuum-packaging a diaper within an interior space of a substantially flexible, substantially air impermeable packaging material such that the diaper is compressed and retained in the compressed condition.

66. The method of claim 65, wherein said vacuum-packaging comprises locating the diaper within an interior space of the packaging material, creating a negative pressure within the interior space, and hermetically sealing the diaper within the interior space at the negative pressure.

67. The method of claim 65, further comprising folding the diaper prior to vacuum-packaging the diaper.

68. The method of claim 65, further comprising rolling the diaper prior to vacuum-packaging the diaper.

69. A method of individually packaging a diaper, comprising:
(a) compressing the diaper from a first condition to a second, compressed condition, and
(b) retaining the diaper in the second, compressed condition with a pressure differential that acts upon a substantially flexible, substantially air impermeable material to maintain the diaper in the compressed condition, the pressure differential being created by,
(i) locating the diaper in an interior space of a substantially flexible material,
(ii) creating a negative pressure within the interior space, and
(iii) hermetically sealing the diaper at the negative pressure within the flexible material.

70. The method of claim 69, wherein said steps (a) and (b) are accomplished by vacuum-packaging the diaper within the interior space of the substantially flexible material.

71. The method of claim 69, wherein the diaper is packaged such that the packaged diaper is dimensioned to fit within a pocket of an article of clothing on one's person.

72. The method of claim 69, wherein the flexible packaging material comprises a thermoplastic shrink-wrap material.

73. The method of claim 69, further comprising reconfiguring the diaper prior to said step of compressing the diaper in said step (a).

74. The method of claim 73, wherein said step of reconfiguring the diaper includes folding the diaper.

75. The method of claim 74, wherein said folding includes forming three crosswise folds in the diaper.

76. The method of claim 73, wherein said step of reconfiguring the diaper includes rolling the diaper such that the diaper is cylindrical in shape.

77. The method of claim 69, wherein said step of compressing of the diaper includes reducing two dimensions of the group of height, width, and length of the diaper.

78. The method of claim 77, wherein the two dimensions are reduced without folding or rolling of the diaper during said step of compressing the diaper.

79. The method of claim 78, wherein at least one of the reduced dimensions is reduced by at least 30%.

80. The method of claim 69, wherein said step of compressing the diaper includes reducing two dimensions of the group of width, thickness, and diameter of the diaper.

81. The method of claim 80, wherein the two dimensions of the diaper are reduced without folding or rolling of the diaper during said step of compressing the diaper.

82. The method of claim 81, wherein one of the reduced dimensions is reduced by at least 30%.

83. The method of claim 69, wherein said step of compressing the diaper includes compressing the diaper such that a volume of the diaper is reduced by at least 30%.

84. The method of claim 69, wherein said step of compressing the diaper includes compressing the diaper such that a volume of the diaper is reduced by 50%.

85. A method of individually packaging a diaper, comprising vacuum-packaging a diaper such that the diaper is compressed and retained in the compressed condition, the diaper being disposed in a normal condition in which the diaper is unstressed and uncompressed at the time that the diaper is vacuum-packaged.

86. The method of claim 85, wherein the diaper is vacuum-packaged within an interior space of a substantially flexible, substantially air impermeable packaging material and is the only diaper contained within an interior space of the packaging material.

87. The method of claim 85, wherein said vacuum-packaging comprises locating the diaper within an interior space of the packaging material, creating a negative pressure within the interior space, and hermetically sealing the diaper within the interior space at the negative pressure.

88. A method of individually packaging a diaper, comprising:
(a) compressing the diaper from a first, generally uncompressed and unstressed condition to a second, compressed condition, and
(b) retaining the diaper in the second, compressed condition by,
(i) locating the diaper in an interior space of a substantially flexible material,
(ii) creating a negative pressure within the interior space, and
(iii) hermetically sealing the diaper at the negative pressure within the flexible material.

89. The method of claim 88, wherein both of said steps (a) and (b) are performed by vacuum-packaging the diaper within the interior space of the substantially flexible material.

90. The method of claim 88, wherein the flexible packaging material comprises a thermoplastic shrink-wrap material.

91. The method of claim 88, wherein said step of compressing the diaper includes reducing at least two of height, width, and length dimensions of the diaper.

92. The method of claim 91, wherein at least two dimensions of the group of the height, width, and length of the diaper are reduced without folding or rolling of the diaper during said step of compressing the diaper.

93. The method of claim 92, wherein one of the reduced dimensions is reduced by at least 30%.

94. The method of claim 88, wherein said step of compressing the diaper includes compressing the diaper such that a volume of the diaper is reduced by at least 30%.

95. The method of claim 88, wherein said step of compressing the diaper includes compressing the diaper such that a volume of the diaper is reduced by 50%.

96. A diaper packaged according to the method of claim 88.

97. A method of individually packaging a diaper, comprising:
(a) reconfiguring a diaper from a first configuration to a second configuration; and
(b) compressing the diaper to, and retaining the diaper in, a compressed condition by vacuum-packaging of the reconfigured diaper.

98. The method of claim 97, wherein said vacuum-packaging of the reconfigured diaper comprises the steps of,
(i) locating the diaper in an interior space of a substantially flexible material,
(ii) creating a negative pressure within the interior space, and
(iii) hermetically sealing the diaper at the negative pressure in the interior space of the flexible material.

99. The method of claim 98, wherein said packaged diaper is the only diaper contained within the interior space of the flexible material.

100. The method of claim 97, wherein the diaper is packaged such that the packaged diaper is dimensioned to fit within a pocket of an article of clothing on one's person.

101. The method of claim 97, wherein the flexible packaging material comprises a thermoplastic shrink-wrap material.

102. The method of claim 97, wherein said step (b) is performed after said step (a).

103. The method of claim 97, wherein said step of reconfiguring the diaper includes folding the diaper.

104. The method of claim 103, wherein said folding includes forming three crosswise folds in the diaper.

105. The method of claim 104, wherein said three crosswise folds are about evenly spaced along a length of the diaper between opposed ends of the diaper.

106. The method of claim 97, wherein said step of reconfiguring the diaper includes rolling the diaper such that the reconfigured diaper is cylindrical in shape.

107. The method of claim 97, wherein said step of compressing the diaper includes reducing at least two of height, width, and length dimensions of the diaper.

108. The method of claim 107, wherein at least two of the height, width, and length dimensions of the diaper are reduced without folding or rolling of the diaper during said step of compressing the diaper.

109. The method of claim 108, wherein at least one of the reduced dimensions is reduced by at least 30%.

110. The method of claim 97, wherein said step of compressing the diaper includes reducing at least two of width, thickness, and diameter dimensions of the diaper.

111. The method of claim 110, wherein two dimensions of the group of the width, thickness, and diameter of the diaper are reduced without folding or rolling of the diaper during said step of compressing the diaper.

112. The method of claim 111, wherein one of the reduced dimensions is reduced by at least 30%.

113. The method of claim 97, wherein said step of compressing the diaper includes compressing the diaper such that a volume of the diaper is reduced by at least 30%.

114. The method of claim 97, wherein said step of compressing the diaper includes compressing the diaper such that a volume of the diaper is reduced by 50%.

115. A set of compact absorbent articles, the set comprising:
(a) a packaging including a plurality of compartments; and
(b) a plurality of absorbent articles each individually vacuum sealed within a respective compartment in a compressed condition.

116. The set of claim 115, wherein the packaging includes seams extending between each of the compartments whereby a vacuum sealed absorbent article may be separated from the other absorbent articles of the packaging.

117. The set of claim 116, wherein each seam comprises a line of perforations in a material of the packaging.

118. The set of claim 115, wherein the packaging is substantially flexible whereby the packaging may be folded back upon itself.

119. The set of claim 115, wherein each vacuum sealed absorbent article is disposed in a rolled configuration.

120. The set of claim 115, wherein each vacuum sealed absorbent article is disposed in a folded configuration.

121. The set of claim 115, wherein a volume of at least one of said plurality of vacuum sealed absorbent articles in said compressed condition is reduced relative to a volume of said absorbent article when uncompressed, the reduction being at least 30%.

122. The set of claim 115, wherein a volume of at least one of said plurality of vacuum sealed absorbent articles in said compressed condition is reduced relative to a volume of said absorbent article when uncompressed, the reduction being at least 50%.

123. The set of claim 115, wherein two dimensions of at least one of said plurality of vacuum sealed absorbent articles in said compressed condition is reduced relative to the corresponding dimensions of said absorbent article when uncompressed, the reduction being at least 30%.

124. The set of claim 115, wherein one of said plurality of vacuum sealed absorbent articles consists of a disposable diaper dimensioned to fit a child.

125. The set of claim 115, wherein one of said plurality of vacuum sealed absorbent articles consists of a disposable diaper dimensioned to fit an adult.

126. The set of claim 115, wherein one of said plurality of vacuum sealed absorbent articles consists of training pants.

127. The set of claim 115, wherein one of said plurality of vacuum sealed absorbent articles consists of a sanitary napkin.

128. A portable set of compact absorbent articles, comprising:
(a) a carrier including at least one compartment; and
(b) at least one container received within the compartment of the carrier, each container including therein an absorbent article that is individually vacuum-packaged.

129. The set of claim 128, wherein the vacuum-packaged absorbent article included in a container is disposed in a rolled configuration.

130. The set of claim 128, wherein the vacuum-packaged absorbent article included in a container is disposed in a folded configuration.

131. The set of claim 128, wherein at least one container is rectangular in shape.

132. The set of claim 128, wherein at least one compartment of the carrier is rectangular in shape.

133. A set of compact disposable diapers, comprising:
(a) a carrier; and
(b) a plurality of containers received within the carrier, each container including therein a disposable diaper that is individually vacuum-packaged.

134. The set of claim 133, wherein the vacuum-packaged diaper included in a container is disposed in a rolled configuration.

135. The set of claim 133, wherein the vacuum-packaged diaper included in a container is disposed in a folded configuration.

136. The set of claim 133, wherein at least one container is rectangular in shape.

137. The set of claim 133, wherein at least one compartment of the carrier is rectangular in shape.

138. The set of claim 133, wherein at least one vacuum-packaged diaper comprises a diaper retained in a compressed condition by a pressure differential acting across a flexible packaging material.

139. The set of claim 138, wherein a volume of said diaper retained in said compressed condition is reduced relative to a volume of said diaper when uncompressed, the reduction being at least 30%.

140. The set of claim 138, wherein a volume of said diaper retained in said compressed condition is reduced relative to a volume of said diaper when uncompressed, the reduction being at least 50%.

141. The set of claim 138, wherein two dimensions of said diaper retained in said compressed condition is reduced relative to corresponding dimensions of said diaper when uncompressed, the reduction being at least 30%.

142. The set of claim 133, wherein each diaper is dimensioned to fit a child.

143. The set of claim 133, wherein each diaper is dimensioned to fit an adult.

144. An individually packaged compressible absorbent article that is compact and portable, comprising a compressible absorbent article vacuum-packaged within an interior space of a substantially flexible, substantially air impermeable material such that said absorbent article is maintained in a compressed condition, wherein the absorbent article comprises a resilient and elastic absorptive material.

145. A packaged compressible absorbent article that is compact and portable, comprising a compressible absorbent article vacuum-packaged within an interior space of a substantially flexible, substantially air impermeable material, the vacuum-packaged compressible absorbent article maintained in a compressed condition within said packaging and dimensioned to fit within the pocket of an article of clothing to be worn on one's person, wherein the absorbent article comprises a resilient and elastic absorptive material.

146. An individually packaged compressible absorbent article that is compact and portable, comprising:
(a) packaging made from a substantially flexible, substantially air impermeable material; and
(b) a compressible absorbent article contained within said packaging in a compressed condition, wherein the absorbent article comprises a resilient and elastic absorptive material; and
(c) wherein said packaging is hermetically sealed and a negative pressure exists within said packaging such that said compressible absorbent article is maintained in said compressed condition.

147. The packaged compressible absorbent article of claim 146, wherein said compressible absorbent article is the only compressible absorbent article contained within said packaging.

148. The packaged compressible absorbent article of claim 146, wherein said compressible absorbent article is dimensioned to fit within the pocket of an article of clothing on one's person.

149. The packaged compressible absorbent article of claim 146, wherein said compressible absorbent article is dimensioned to fit within a pocket of a jacket that is worn on one's person.

150. The packaged compressible absorbent article of claim 146, wherein said material comprises a thermoplastic shrink-wrap material.

151. The packaged compressible absorbent article of claim 146, wherein said compressible absorbent article is disposed within said packaging in a folded arrangement and has three crosswise folds.

152. The packaged compressible absorbent article of claim 151, wherein said three crosswise folds are about evenly spaced along a length of the compressible absorbent article between opposed ends of the compressible absorbent article.

153. The packaged compressible absorbent article of claim 146, wherein said compressible absorbent article is disposed within said packaging in a rolled arrangement.

154. The packaged compressible absorbent article of claim 146, wherein a volume of said compressible absorbent article in said compressed condition is reduced relative to a volume of said compressible absorbent article when uncompressed.

155. The packaged compressible absorbent article of claim 154, wherein the reduction is by at least 30%.

156. The packaged compressible absorbent article of claim 154, wherein the reduction is by at least 50%.

157. The packaged compressible absorbent article of claim 146, wherein at least two of height, width, and length of said compressible absorbent article in said compressed condition are reduced relative to the corresponding height, width, and length of said compressible absorbent article when uncompressed.

158. The packaged compressible absorbent article of claim 157, wherein the reduction is by at least 30%.

159. The packaged compressible absorbent article of claim 146, wherein the compressible absorbent article consists of a disposable diaper dimensioned to fit a child.

160. The packaged compressible absorbent article of claim 146, wherein the compressible absorbent article consists of a disposable diaper dimensioned to fit an adult.

161. The packaged compressible absorbent article of claim 146, wherein the compressible absorbent article consists of training pants.

162. The packaged compressible absorbent article of claim 146, wherein the compressible absorbent article consists of a sanitary napkin.

163. A method of folding a disposable diaper for packaging, comprising folding the diaper along three fold lines, each fold line generally extending in a transverse direction, wherein the diaper is folded such that the diaper is disposed in a generally “M” shaped folded arrangement with a first transverse end edge of the diaper being disposed on the same side of the diaper as a second transverse end edge of the diaper.

164. A method of packaging a diaper, comprising the steps of:
(a) folding the diaper along three fold lines, each fold line generally extending in a transverse direction, wherein the diaper is folded such that the diaper is disposed in a generally “M” shaped folded arrangement with a first transverse end edge of the diaper being disposed on the same side of the diaper as a second transverse end edge of the diaper; and
(b) vacuum-packaging the diaper while disposed in the folded arrangement.

165. The method of claim 164, wherein the vacuum-packaging of the diaper includes compressing the diaper such that a volume of the diaper is reduced by at least 30%.

166. The method of claim 164, wherein the vacuum-packaging of the diaper includes compressing the diaper such that a volume of the diaper is reduced by at least 50%.

167. The method of claim 164, wherein the vacuum-packaging of the diaper includes reducing a dimension of the folded diaper by at least 30%.

168. The packaged compressible absorbent article of claim 164, wherein the compressible absorbent article consists of a disposable diaper dimensioned to fit a child.

169. The packaged compressible absorbent article of claim 164, wherein the compressible absorbent article consists of a disposable diaper dimensioned to fit an adult.

170. The packaged compressible absorbent article of claim 164, wherein the compressible absorbent article consists of training pants.
